(19) United States
(12) Patent Application Publication
(10) Pub. No.: US 2005/0043274 A1
(43) Pub. Date: Feb. 24, 2005

(54) Pharmaceutical compositions and methods for lowering blood pressure and pulse rate

(76) Inventor: Howard Murad, Marina del Rey, CA (US)

Correspondence Address: MORGAN LEWIS & BOCKIUS LLP, 1111 PENNSYLVANIA AVENUE NW, WASHINGTON, DC, 20004, US

(21) Appl. No.: 10/909,344

(22) Filed: Aug. 3, 2004

Related U.S. Application Data
(60) Provisional application No. 60/496,919, filed on Aug. 22, 2003.

(51) Int. Cl. A61K 31/7008; A61K 31/401; A61K 31/375; A61K 31/198

(52) U.S. Cl. 514/62; 514/561; 514/474; 514/423; 514/562; 514/564

(57) ABSTRACT

A pharmaceutical composition comprising (1) a compound that is converted to a glycosaminoglycan in a patient; (2) a primary antioxidant component; (3) at least one amino acid component; and (4) one or more of lecithin, phosphatidyl choline, or choline and methods for lowering blood pressure and/or pulse rate in a patient by administering to the patient the pharmaceutical composition.

### CROSS REFERENCE TO RELATED APPLICATION

This application claims the benefit of provisional application No. 60/496,919, filed Aug. 22, 2003, the contents of which are incorporated herein in their entirety.

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### TECHNICAL FIELD

This application relates to compositions and methods to lower blood pressure and/or pulse rate in a patient.

### BACKGROUND OF THE INVENTION

Arterial hypertension is an elevation of systolic and/or diastolic blood pressure (see e.g., Merck Manual, 16th edition, 1992, pages 413-429). Hypertension is defined as systolic blood pressure of 140 millimeters of mercury or more and/or diastolic blood pressure of 90 millimeters of mercury or more. Primary or essential hypertension is of unknown etiology, although heredity appears to predispose individuals to hypertension. More than 50 million persons in the United States have hypertension.

Primary hypertension is asymptomatic until complications develop. Symptoms and signs are non-specific and arise from complications in target organs. Complications include, for example, left ventricular failure, atherosclerotic heart disease, retinal hemorrhages, exudates, papilledema, vascular accidents, cerebrovascular insufficiency with or without stroke, and renal failure.

There is no cure for primary hypertension, but therapy can modify its course. In cases of mild hypertension, non-drug therapies such as weight reduction, restriction of dietary sodium to less than 2000 milligrams per day, limiting alcohol intake to less than one ounce of ethanol per day, and prudent exercise can make drug therapy unnecessary. However, when complications are present or impending, or when the diastolic blood pressure is greater than 95 mm, it is recommended that drug therapy should not be deferred. The goal of drug therapy is to reduce the blood pressure to normal, i.e. 140 mm/90 mm or less.

Antihypertensive drugs include four major classes of agents, identified as diuretics, beta-blockers, calcium antagonists, and angiotensin-converting enzyme (ACE) inhibitors. Examples of drugs within these classes include the following: diuretics, such as furosemide (Laxix) and hydrocholrothizide (HydroDIURIL); beta-blockers, such as atenolol (Tenormin), metoprolol (Lopressor), and propranolol (Inderal); calcium antagonists, such as amlodipine (Norvasc) and diltizem (Cardizem); and antiotensin-converting enzyme inhibitors, such as captopriol (Capoten), enalapril (Vasotec), and lisinopril (Prinivil). The choice of antihypertensive drug is based on the patient's age, race, and the presence or absence of particular complications and conditions associated with high blood pressure.

U.S. Pat. No.4,725,588 to Snyder et al. discloses a method of lowering blood pressure using alkyl phospholipid antihypertensive agents.

U.S. Pat. No.5,008,411 to Coffen et al. discloses gycidic acid esters allegedly useful as calcium channel blockers and accordingly useful as agents for lowering blood pressure.

U.S. Pat. No.5,143,915 to Rovnyak et al. discloses a specific calcium antagonist useful for reducing blood pressure.

U.S. Pat. No.5,708,029 to Vanmoor discloses a method for determining the effectiveness of an agent for the relief of high blood pressure.

U.S. Pat. No. 5,804,594 to Murad discloses pharmaceutical compositions for improving wrinkles and other skin conditions. The compositions include a sugar compound that is converted to glycosaminoglycan in a patient, an amino acid component, a primary antioxidant component, and a transition metal.

Despite attempts in the pharmaceutical industry to develop compositions to reduce blood pressure and pulse rate, no widely accepted solution exists. Moreover, many of the proposed solutions have unpleasant and even harmful side effects. There is thus a need for improved compositions and methods to lower blood pressure and pulse rate.

### SUMMARY OF THE INVENTION

This present invention encompasses a pharmaceutical composition comprising a compound that is converted to a glycosaminoglycan in a patient; an amino acid component; a primary antioxidant; and one or more of lecithin, phosphatidyl choline, or choline. The invention also relates to methods for lowering blood pressure and pulse rate in a patient, which comprise administering to the patient a therapeutically effective amount of the pharmaceutical composition.

### DETAILED DESCRIPTION OF THE PREFERRED EMBODIMENTS

The compositions of the invention comprise (i) a compound that is converted to a glycosaminoglycan in a patient; (ii) an amino acid component; (iii) a primary antioxidant; and (iv) one or more of lecithin, phosphatidyl choline, or choline. The compositions when administered to a patient reduce blood pressure and pulse rate. In one embodiment the patient is a mammal. In another embodiment, the patient is a human.

The composition contains at least one compound that is converted to a glycosaminoglycan in the patient, and preferably just one such compound, present in an amount ranging from about 5 to 50 weight percent, preferably in an amount ranging from about 10 to 40 weight percent, and more preferably in an amount ranging from about 15 to 30 weight percent of the composition.

The primary antioxidant component is present in an amount ranging from about 5 to 50 weight percent, preferably in an amount ranging from about 10 to 40 weight percent, and more preferably in an amount ranging from about 15 to 30 weight percent of the composition.

The amino acid component is present in an amount ranging from about 8 to 60 weight percent, preferably in an amount ranging from about 15 to 50 weight percent, and more preferably in an amount ranging from about 20 to 40 weight percent of the composition.

One or more of lecithin, phosphatidylcholine, or choline is present in an amount ranging from about 0.5 to 50 weight percent, preferably in an amount ranging from about 1 to 40 weight percent, and more preferably in an amount ranging from about 2 to 30 weight percent of the composition.

The compound used in the compositions and methods of the invention that is converted to a glycosaminoglycan can be any compound that is converted to a glycosaminoglycan in the patient, typically in the patient's bloodstream. In one embodiment, the compound that is converted to a glycosaminoglycan in the patient is a sugar or a derivative of a sugar. Suitable derivatives of a sugar include, but are not limited to, glucosamine, carbonyl esters of sugars, phosphenyl esters of sugars, and sulfonyl esters of sugars. Glycosaminoglycans are high molecular weight polysaccharides that contain amino sugars and often form complexes with proteins (see e.g., IUPAC Compendium of Chemical Terminology, 2^{nd} Edition, 1997, http://www.chemsoc.org/chembytes/goldbook/). In one embodiment, the glycosaminoglycan is N-acetylglucosamine, or a pharmaceutically acceptable salt or ester thereof, but preferably is simply N-acetylglucosamine. N-acetylglucosamine has the formula: 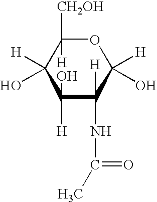
N-acetylglucosamine is typically present in an amount ranging from about 5 to 30 weight percent, preferably from about 8 to 27 weight percent, and more preferably from about 12 to 24 weight percent of the pharmaceutical composition. A unit dose of N-acetylglucosamine is typically about 40 mg to 250 mg, preferably about 60 mg to 200 mg, and more preferably about 100 mg to 200 mg.

The primary antioxidant is typically a vitamin C source and preferably is ascorbic acid, or a pharmaceutically acceptable salt or ester thereof, and more preferably is ascorbyl palmitate, dipalmitate L-ascorbate, sodium L-ascorbate-2-sulfate, or an ascorbic salt, such as sodium, potassium, or calcium ascorbate, or mixtures thereof. When oral formulations of the pharmaceutical composition are used, it is preferred that a non-acidic form of vitamin C be used to reduce the stomach irritation that may occur when using an acidic form. The vitamin C source is present in the pharmaceutical composition in an amount ranging from about 5 to 50 weight percent, preferably from about 10 to 40 weight percent, and more preferably from about 15 to 30 weight percent. A unit dose of the vitamin C source is typically about 40 mg to 400 mg, preferably about 60 mg to 300 mg, and more preferably about 80 to 150 mg. Vitamin C is also approved by the FDA and has wide consumer acceptance, and can be used in amounts as high as 10,000 mg, if desired.

The pharmaceutical composition also includes at least one amino acid. Preferably two or more amino acids are used in combination. Either the L- or D- forms of amino acids are acceptable. Lysine and proline are the preferred amino acids and are advantageously used in combination. Cysteine, methionine or other amino acids can also be used, if desired. In one embodiment, the amino acids are included in a soluble form such as the hydrochloride salt, i.e., L-Lysine hydrochloride. The amino acids are present in a combined amount ranging from about 8 to 60 weight percent, preferably from about 15 to 50 weight percent, and more preferably from about 20 to 40 weight percent. A unit dose for each amino acid is typically about 35 mg to 200 mg each, preferably about 50 mg to 150 mg each, and more preferably about 70 mg to 120 mg.

Useful forms of amino acids include, but are not limited to, the following: a cysteine source, preferably N-acetyl cysteine, present in an amount ranging from about 1 to 10 weight percent, preferably from about 2 to 8 weight percent, and more preferably from about 3 to 6 weight percent of the pharmaceutical composition and a methionine source, preferably L-selenomethionine, present in an amount ranging from about 0.1 to 5 weight percent, preferably from about 0.2 to 3 weight percent, and more preferably from about 0.3 to 1 weight percent of the composition, wherein the selenium component is between about 0.1 to 3 weight percent of the methionine source.

The pharmaceutical composition also includes one or more of lecithin, phosphatidyl choline, or choline.

Phosphatidyl choline has the formula: 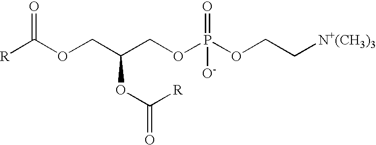
wherein —OC(O)R is derived from a fatty acid. A unit dose for phosphatidyl choline is typically from about 4 mg to about 400 mg, preferably from about 8 mg to 300 mg, and more preferably from about 16 mg to 230 mg. When phosphatidyl choline is included as part of the composition it is typically present in an amount of from about 0.5 to 50 weight percent, preferably from about 1 to about 40 weight percent, and more preferably from about 2 to 30 weight percent of the composition.

Lecithin is a mixture of phosphatidyl choline molecules having a variety of —OC(O)R groups. A unit dose for lecithin is typically from about 4 mg to about 400 mg, preferably from about 8 mg to 300 mg, and more preferably from about 16 mg to 230 mg. When lecithin is included as part of the composition it is typically present in an amount ranging from about 0.5 to 50 weight percent, preferably from about 1 to about 40 weight percent, and more preferably from about 2 to about 30 weight percent of the composition.

Choline has the formula: 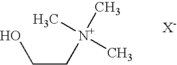
wherein X⁻ is a pharmaceutically acceptable anion. Representative pharmaceutically acceptable anions, X⁻, include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)). A unit dose for choline is typically from about 4 mg to about 400 mg, preferably from about 8 mg to about 300 mg, and more preferably from about 16 mg to about 230 mg. When choline is included as part of the composition it is typically present in an amount of from about 0.5 to about 50 weight percent, preferably from about 1 to about 40 weight percent, and more preferably from about 2 to about 30 weight percent of the composition.

In an embodiment of the invention, the pharmaceutical composition further includes a catechin-based preparation, such as a proanthanol or proanthocyanidin, optionally in combination with glucosamine or a pharmaceutically acceptable salt or ester thereof or chondroitin or a pharmaceutically acceptable salt or ester thereof.

The catechin-based preparation, similar to vitamin C, inhibits elastase and collagenase, which is an enzyme that attacks elastic tissue and collagen. The catechin-based preparation is preferably a proanthanol or proanthocyanidin, more preferably a proanthocyanidin, and most preferably grape seed extract. These compounds are considered to be secondary antioxidants, because they are present in lesser amounts than the primary antioxidant. The catechin-based preparation is present in an amount ranging from about 0.5 to 5 weight percent, preferably from about 0.6 to 3 weight percent, and more preferably from about 0.7 to 2 weight percent of the pharmaceutical composition.

The glucosamine or a pharmaceutically acceptable salt or ester thereof, and the chondroitin or a pharmaceutically acceptable salt or ester thereof are each present in an amount ranging from about 3 to 17 weight percent, preferably from about 4 to 12 weight percent each, and more preferably from about 5 to 8 weight percent each of the pharmaceutical composition. The glucosamine component preferably is present as a sulfate or succinate salt, and more preferably is D-glucosamine sulfate, wherein the glucosamine is preferably present as about 60 to 90 weight percent of the salt. The glucosamine component contributes to the formation of glycosoaminoglycans in the skin.

The chondroitin component preferably is present as a sulfate or succinate salt, and more preferably is chondroitin sulfate, wherein the chondroitin is preferably present as about 65 to 95 weight percent of the salt.

In another embodiment, one or more optional additives are included in the pharmaceutical composition, such as a vitamin E source, a vitamin B₃ source, quercetin powder, pyridoxal 5 phosphate-Co B₆, and a vitamin A source. The vitamin E preferably is a sulfate or succinate vitamin E complex, and more preferably is D-alpha tocopheryl acid succinate. The vitamin E source is present in an amount ranging from about 1 to 15 weight percent, preferably from about 2 to 12 weight percent, and more preferably from about 3 to 10 weight percent of the composition. In any event, no more than 1,500 IU of a Vitamin E source should be ingested per day, as Vitamin E becomes toxic at higher doses.

The vitamin B₃ source preferably is niacinamide, and the source is present in an amount ranging from about 0.5 to 15 weight percent, preferably from about 1 to 12 weight percent, and more preferably from about 1.5 to 10 weight percent of the composition.

The vitamin A source preferably is vitamin A palmitate, and is present in an amount ranging from about 0.1 to 5 weight percent, preferably from about 0.2 to 3 weight percent, and more preferably 0.3 to 1 weight percent of the composition. Vitamin A is toxic at high levels, such that no more than 400,000 IU should be cumulatively ingested per day for greater than six months.

The quercetin powder is quercetin dihydrate, and is typically present in an amount ranging from about 0.5 to 15 weight percent, preferably from about 1 to 12 weight percent, and more preferably from about 1.5 to 10 weight percent of the composition.

The pyridoxal 5 phosphate-Co B₆, also known as P-5-P monohydrate, is typically present in an amount ranging from about 0.1 to 5 weight percent, preferably from about 0.2 to 3 weight percent, and more preferably from about 0.3 to 1 weight percent of the composition.

The phrase “therapeutically effective amount” means that amount of the pharmaceutical composition that, in the absence of other effects that lower blood pressure or pulse rate, lowers the blood pressure or pulse rate in a patient by at least two percent.

The magnitude of a prophylactic or therapeutic dose of the composition in the acute or chronic management of high blood pressure or pulse rate will vary with the severity of the condition to be treated and the route of administration. The dose, and perhaps the dose frequency, will also vary according to the age, body weight, and response of the individual patient. In general, the total daily dose range, for the conditions described herein, is from about 10 mg to about 20,000 mg administered in single or divided doses orally, topically, transdermally, or locally by inhalation. For example, a preferred oral daily dose range should be from about 10 mg to 20,000 mg, more preferably about 2,000 mg to 16,000 mg, and most preferably about 6,000 mg to 10,000 mg of the active components (i.e., excluding excipients and carriers).

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know how and when to interrupt, adjust, or terminate therapy in conjunction with individual patient response.

The term “unit dose” is meant to describe a single dose. About 1 to 10 unit doses of the present invention are typically administered per day, preferably about 2 to 6 unit doses per day, and more preferably about 4 unit doses per day. In one embodiment, a single unit dose is administered daily.

The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of inorganic bases, for potential salt formation with compounds of the invention, include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine.

Any suitable route of administration may be employed to administer the compositions of the invention to a patient. Suitable routes include, for example, oral, rectal, parenteral, intravenous, topical, transdermal, subcutaneous, and intramuscular. Although any suitable route of administration may be employed for providing the patient with an effective dosage of the composition according to the methods of the present invention, oral administration is preferred.

Suitable dosage forms include solids such as tablets, capsules, and troches; liquids such as dispersions, suspensions, elixirs, and solutions; patches; suppositories; aerosols; and the like. Oral dosage forms are preferred. Solid oral preparations are preferred over liquid oral preparations. Because of their ease of administration, tablets and capsules represent the most advantageous oral dosage unit form, in which case solid pharmaceutical carriers are employed. The most preferred oral solid preparations are tablets. If desired, tablets may be coated by standard aqueous or non-aqueous techniques.

The pharmaceutical compositions of the present invention include the active ingredients described above, and may also contain pharmaceutically acceptable carriers, excipients, and the like, and optionally, other therapeutic ingredients. Representative carriers and excipients include, but are not limited to, starches, sugars, microcrystalline cellulose, diluents, granulating agents, lubricants, binders, disintegrating agents, and the like, in the case of oral solid preparations (such as powders, capsules, and tablets).

Pharmaceutical compositions for use in the methods of the present invention suitable for oral administration may be presented as discrete units such as capsules, cachets, tablets; aerosol sprays; or a solution or a suspension in an aqueous liquid, a non-aqueous liquid, an oil-in-water emulsion, or a water-in-oil liquid emulsion, each containing a predetermined amount of the active ingredient. Such compositions may be prepared by any of the methods of pharmacy, but all methods include the step of bringing into association the carrier with the active ingredient which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent. Desirably, each unit dose, i.e., tablet, cachet or capsule, contains from about 1 mg to 2,000 mg of the active ingredients, preferably about 200 mg to 1,600 mg, and more preferably about 600 mg to 1,000 mg by weight of the composition.

In addition to the common dosage forms set out above, the compound for use in the methods of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference.

### EXAMPLES

The invention is further defined by reference to the following examples describing in detail the preparation of the compositions used in the methods of the present invention, as well as their utility. The examples are representative, and they should not be construed to limit the scope of the invention.

### Example 1

### Capsules

A large number of unit capsules are prepared by filling standard two-piece hard gelatin capsules each with the desired amount of powdered active ingredient, 150 milligrams of lactose, 50 milligrams of cellulose, and 6 milligrams magnesium stearate.

### Example 2

### Soft Gelatin Capsules

A mixture of active ingredient in a digestible oil such as soybean oil, lecithin, cottonseed oil or olive oil is prepared and injected by means of a positive displacement pump into gelatin to form soft gelatin capsules containing the desired amount of the active ingredient. The capsules are washed and dried for packaging.

### Example 3

### Tablets

A large number of tablets were prepared by conventional procedures so that each dosage unit included the ingredients listed in the following table.

| | Weight | | Chemical or |
|---|---|---|---|
| | Percent | Amount | Scientific |
| Ingredient | (% w/w) | (mg) | Name (if different) |
| N-Acetylglucosamine | 17.7 | 140 | N-Acetyl |
| | | | D-Glucosamine |
| Vitamin C (81.2% Ascorbic | 15.6 | 123.2 | |
| Acid) | | | |
| L-Lysine (80%) | 12.6 | 100 | L-Lysine |
| | | | hydrochloride |
| L-Proline | 11.3 | 90 | |
| D-Glucosamine Sulfate (75%) | 6.7 | 53.3 | |
| Chondroitin Sulfate (80%) | 6.3 | 50 | |
| Vitamin E Succinate | 4.4 | 39.7 | D-alpha tocopheryl |
| | | | acid succinate |
| N-Acetyl Cysteine | 3.8 | 30 | |
| Lecithin | 2.9 | 23.1 | |
| Vitamin B₃ Niacinamide | 2.5 | 20 | Niacinamide |
| Quercetin Powder | 2.5 | 20 | Quercetin dihydrate |
| Grape Seed Extract | 0.93 | 7.5 | Proanthocyanidin |
| Pyridoxal 5 Phosphate-Co B₆ | 0.62 | 5 | P-5-P monohydrate |
| Selenomethionine (0.5%) | 0.52 | 4 | L-selenomethionine |
| Vitamin A Palmitate | 0.52 | 4 | |
| Copper Sebacate (14%) | 0.41 | 2.9 | |
| Red beet root powder | 6.3 | 50 | Beta vulgaris rubra |
| Stearic acid | 1.55 | 12 | |
| Sorbitol | 1.34 | 11 | |
| Acdisol | 0.41 | 3 | Microcrystalline |
| | | | cellulose |
| Coconut oil | 0.1 | 1 | Magnesium stearate |
| Syloid | 0.1 | 1 | Silicon dioxide |
| | | | (amorphous) |
| Total | 100 | 790.7 | |

These tablets are an example of an embodiment of a unit dose according to the present invention.

### Example 4

### Case Study

In one case study, a 70 year old man who had high blood pressure was treated with Wet Suit Supplement®, a composition comprising Vitamin C, Vitamin E (acetate), Zinc, Manganese, Copper, Selenium, Type II Collagen, Glucosamine Sulfate, Essential Fatty Acids, Dipotassium Phosphate, Choline, L-Lysine, L-Glycine, Aloe Vera Concentrate, Potassium Sulfate, Curcumin (from turmeric), Co Q 10, Pomegranate Extract (5% ellagic acid), Phosphatidylcholine(from lecithin), Cellulose, Vegetable Stearate, Magnesium Stearate, Stearic Acid, Silica, Pharmaceutical Glaze, Talc. After 26 days of treatment, the patient's blood pressure was substantially lower.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference in their entireties.

1. A pharmaceutical composition comprising the following components:
(i) a compound that is converted to a glycosaminoglycan in the patient;
(ii) a primary antioxidant component;
(iii) at least one amino acid component; and
(iv) one or more of lecithin, phosphatidyl choline, or choline.

2. The pharmaceutical composition of claim 1, wherein the compound converted to a glycosaminoglycan in the patient is present in an amount ranging from about 5 to 50 weight percent; the primary antioxidant component is present in an amount ranging from about 5 to 50 weight percent; the amino acid component is present in an amount ranging from about 8 to 60 weight percent; and lecithin, phosphatidyl choline, or choline is present in an amount ranging from about 0.5 to 50 weight percent of the composition.

3. The pharmaceutical composition of claim 1, wherein the compound converted to a glcyosaminoglycan in the patient comprises N-acetylglucosamine or a salt or ester thereof, the primary antioxidant component comprises ascorbic acid or a salt or ester thereof, and the amino acid component comprises at least two amino acids selected from the group consisting of proline, lysine, cysteine, and methionine.

4. The pharmaceutical composition of claim 3, wherein the N-acetylglucosamine is present in an amount ranging from about 5 to 30 weight percent; the ascorbic acid is present in an amount ranging from about 5 to 50 weight percent; the amino acid component comprises lysine and proline, wherein each is present in an amount ranging from about 4 to 25 weight percent.

5. The pharmaceutical composition of claim 1, wherein the composition further comprises a pharmaceutically acceptable carrier or excipient.

6. The pharmaceutical composition of claim 1, further comprising a catechin-based preparation, optionally in combination with a glucosamine or a pharmaceutically acceptable salt or ester thereof or a chondroitin or a pharmaceutically acceptable salt or ester thereof.

7. The pharmaceutical composition of claim 6, wherein the catechin-based preparation is a proanthanol or proanthocyanidin, and the glucosamine and chondroitin are each a sulfate or succinate salt.

8. The pharmaceutical composition of claim 7, wherein the proanthocyanidin is grape seed extract present in an amount ranging from about 0.5 to 5 weight percent, the glucosamine is D-glucosamine sulfate present in an amount ranging from about 3 to 17 weight percent, and the chondroitin is chondroitin sulfate present in an amount ranging from about 3 to 17 weight percent of the composition.

9. The pharmaceutical composition of claim 1, further comprising one or more of a vitamin E source, a cysteine source, a vitamin B₃ source, quercetin dihydrate, pyridoxal 5 phosphate-Co B₆, a methionine source, and a vitamin A source.

10. The pharmaceutical composition of claim 9, wherein the vitamin E source is D-alpha tocopheryl acid succinate present in an amount ranging from about 1 to 15 weight percent, the vitamin B₃ source is niacinamide present in an amount ranging from about 0.5 to 15 weight percent, the vitamin A source is vitamin A palmitate present in an amount ranging from about 0.1 to 5 weight percent, the cysteine source is N-acetyl cysteine present in an amount ranging from about 1 to 10 weight percent, the methionine source is L-selenomethionine present in an amount ranging from about 0.1 to 5 weight percent, the quercetin dihydrate is present in an amount ranging from about 0.5 to 15 weight percent, and the pyridoxal 5 phosphate-Co B₆ is present in an amount ranging from about 0.1 to 5 weight percent of the composition.

11. A method for lowering blood pressure in a patient, which comprises administering to the patient a therapeutically effective amount of the pharmaceutical composition of claim 1.

12. The method of claim 11, wherein the pharmaceutical composition is administered orally.

13. The method of claim 12, wherein the pharmaceutical composition is administered as a tablet or capsule comprising about 1 mg to 2,000 mg of the pharmaceutical composition.

14. The method of claim 13, wherein the tablet or capsule comprises about 200 mg to 1,600 mg of the pharmaceutical composition.

15. The method of claim 14, wherein the tablet or capsule comprises about 600 mg to 1,000 mg of the pharmaceutical composition.

16. The method of claim 11, further comprising administering at least one additional agent for lowering blood pressure.

17. A method for lowering pulse rate in a patient, comprising administering to the patient a therapeutically effective amount of the pharmaceutical composition of claim 1.

18. The method of claim 17, wherein the pharmaceutical composition is administered orally.

19. The method of claim 18, wherein the pharmaceutical composition is administered as a tablet or capsule comprising about 1 mg to 2,000 mg of the pharmaceutical composition.

20. The method of claim 19, wherein the tablet or capsule comprises about 200 mg to 1,600 mg of the pharmaceutical composition.

21. The method of claim 20, wherein the tablet or capsule comprises about 600 mg to 1,000 mg of the pharmaceutical composition.

22. The method of claim 17, further comprising administering at least one additional agent for lowering pulse rate.
